(19) United States
(12) Patent Application Publication
(10) Pub. No.: US 2024/0141420 A1
(43) Pub. Date: May 2, 2024

(54) PARALLEL DETECTION AND QUANTIFICATION OF NUCLEIC ACID BASED MARKERS

(71) Applicant: ToDx Inc., Tel Aviv (IL)

(72) Inventors: Noam Kedem Mamet, Tel Aviv (IL); Gil Harari, Hod Hasharon (IL)

(21) Appl. No.: 18/280,849

(22) PCT Filed: Mar. 9, 2022
(86) PCT No.: PCT/IB22/00102

§ 371 Date: Sep. 7, 2023

Related U.S. Application Data
(60) Provisional application No. 63/158,365, filed on Mar. 9, 2021.

(51) Int. Cl. C12Q 1/6855; B01L 3/00; C12Q 1/6837; C12Q 1/6876

(52) U.S. Cl. CPC C12Q 1/6855; B01L 3/502761; C12Q 1/6837; C12Q 1/6876; B01L 2200/0647; B01L 2200/16; B01L 2300/0636; B01L 2300/0816; C12Q 2600/16; C12Q 2600/166

(57) ABSTRACT

The present disclosure describes methods and devices for detection and quantification of target polynucleotides in a sample using targeting nucleic acids immobilized to predetermined locations of a surface.

### RELATED APPLICATIONS

This application claims the benefit of priority to U.S. Provisional Patent Application Ser. No. 63/158,365, filed Mar. 9, 2021, which is hereby incorporated by reference in its entirety.

### BACKGROUND

Molecular diagnosis of genetic defects and diseases requires techniques that are capable of detecting small quantities of DNA and/or RNA in a sample, or techniques that are extremely sensitive to detect mutations in such nucleic acids. For example, techniques such as southern blot, polymerase chain reaction (PCR), reverse transcriptase-polymerase chain reaction, and ligase chain reaction have been extensively used to detect microbial and viral pathogens, and to diagnosis of cancers and genetic diseases.

The development of simple, fast and reliable amplification-based assays to detect target nucleic acids will greatly aid molecular diagnosis.

### SUMMARY

Provided herein are methods and devices related to detection and/or quantification of one or more target polynucleotides in a sample.

In some aspects, provided herein is a method of quantifying one or more target polynucleotides in a sample, the method comprising: (a) contacting a sample to a solid surface on which a plurality of targeting nucleic acids (e.g., primers) are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) counting the number of distinct clusters of amplicons for each target polynucleotide at the pre-determined location to quantify the amount of each target polynucleotide in the sample.

In some aspects, provided herein is a method of detecting the presence of one or more target polynucleotides in a sample (if any are present), the method comprising: (a) contacting a sample to a solid surface on which a plurality of targeting nucleic acids (e.g., primers) are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) detecting the position of clusters of amplicons on the solid surface to detect the presence of one or more target polynucleotides in the sample.

In some embodiments, the targeting nucleic acid comprises a primer. In some embodiments, only forward primers or reverse primers specific to the target polynucleotide are immobilized to the solid surface. In some embodiments, other primers that form a primer pair with the immobilized forward primers or reverse primers are contacted to the solid surface prior to step (b). In some embodiments, both forward and reverse primers specific to the one or more target polynucleotides are immobilized to the solid surface. In some embodiments, the primers comprise a set of nested primers for the same target polynucleotide. In some embodiments, one cluster of primers spreads into another cluster of primers within the set of nested primers for the target polynucleotides

In some embodiments, the targeting nucleic acids are uniformly immobilized to the predetermined location of the solid surface. In some embodiments, the targeting nucleic acids are immobilized to the solid surface as an array of targeting nucleic acid clusters, with each targeting nucleic acid cluster located at one of the predetermined locations on the solid surface. In some embodiments, the targeting nucleic acid clusters are spatially separated from each other on the solid surface. In some embodiments, multiple clusters of targeting nucleic acids for the same target polynucleotides are immobilized on the solid surface. In some embodiments, multiple clusters of targeting nucleic acids for detecting more than one target polynucleotides from the same cell, organism, or pathogen are immobilized to the solid surface. In some embodiments, each cluster of targeting nucleic acids comprises at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or more copies of the same targeting nucleic acid or set of targeting nucleic acids specific for the same target polynucleotide. In some embodiments, the targeting nucleic acid cluster is immobilized to a surface area with a size at least 0.01 mm², 0.0198 mm², 0.02 mm², 0.03 mm², 0.04 mm², 0.05 mm², 0.06 mm², 0.07 mm², 0.08 mm², 0.09 mm², 0.1 mm², 0.2 mm², 0.3 mm², 0.4 mm², 0.5 mm², 0.6 mm², 0.7 mm², 0.8 mm², 0.9 mm², 1 mm², 2 mm², 3 mm², 4 mm², 5 mm², 6 mm², 7 mm², 8 mm², 9 mm², or 10 mm². In some embodiments, the cluster of targeting nucleic acid is in a square shape with a side of 141 micron.

In some embodiments, the targeting nucleic acids (e.g., primers) are immobilized to the solid surface via its 5′ end. In some embodiments, the targeting nucleic acids are covalently linked to the solid surface. In some embodiments, the targeting nucleic acids are 5′ DBCO modified and the solid surface is azide functionalized surface. In some embodiments, the targeting nucleic acids are 5′ amine modified and the solid surface is NHS functionalized surface. In some embodiments, the targeting nucleic acids are non-covalently linked to the solid surface. In some embodiments, the targeting nucleic acids are immobilized to the solid surface via passive absorption, streptavidin-biotin interaction, or hybridization. In some embodiments, the 3′end of the targeting nucleic acids are free to elongate. In some embodiments, the targeting nucleic acids are immobilized on a 2-dimensional surface. In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 10⁴/micron², 5×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, or 5×10⁶/micron³. In some embodiments, the targeting nucleic acids are invading primers. In some embodiments, the invading primers are LNA, PNA, PTO, ZNA, invader probe, or INA.

In some embodiments, the solid surface is a slide, a chip, a microwell plate, a well plate, a fluid chamber, a matrix, a plate, a tube, or a fluidic channel.

In some embodiments, the one or more target polynucleotides are DNA. In some embodiments, the one or more target polynucleotides are a single-stranded polynucleotides. In some embodiments, the one or more target polynucleotides are double-strand polynucleotides. In some embodiments, the method further comprises denaturing any polynucleotides in the sample prior to step (b) to generate single-strand polynucleotides. In some embodiments, a bisulfite conversion of the DNA sample occurred before step (a). In some embodiments, the target polynucleotides are RNA. In some embodiments, reverse transcriptase is contacted to the sample before step (b). In some embodiments, the target polynucleotides are linked to a barcode at their 5′ end, 3′ end, or at both ends. In some embodiments, the methods described herein further comprise adding a barcode to the 5′end, 3′ end, or both ends of the target polynucleotides in the sample before step (a).

In some embodiments, a nucleic acid purification step is performed on the sample prior to step (a). In some embodiments, no nucleic acid purification step is performed on the sample prior to step (a). In some embodiments, the step (a) comprises (i) annealing the targeting nucleic acid to the corresponding target polynucleotide in the sample, (ii) washing the solid surface to remove contaminates from the sample, (iii) contacting the solid surface with a reaction mix.

In some embodiments, the sample comprises a reference sequence, and targeting nucleic acids for the reference sequence are immobilized to the solid surface. In some embodiments, the sample is diluted prior to the step (a). In some embodiments, a pool of samples is used in the step (a).

In some embodiments, the amplification process is a semi-solid phase amplification with one end on the solid surface and the other end in suspension. In some embodiments, the amplification process is a solid phase amplification. In some embodiments, the solid phase amplification process is a bridge amplification.

In some embodiments, the amplification process is a stepwise thermal amplification. In some embodiments, the amplification process is a stepwise chemical amplification. In some embodiments, the amplification process is PCR or RT-PCR. In some embodiments, the amplification process is an isothermal amplification. In some embodiments, the isothermal amplification process is TMA, NASBA, LAMP, HIP, HDA, RPA, SDA, or rolling circle amplification. In some embodiments, the step (b) occurs at an ambient temperature. In some embodiments, the step (b) occurs at about 37° C. to about 42° C. In some embodiments, the step (b) occurs at human body temperature. In some embodiments, the step (b) occurs without a mechanical heating device. In some embodiments, heat induced by chemical exothermic reaction at the step (b). In some embodiments, the amplification is non-enzymatic amplification process. In some embodiments, the non-enzymatic amplification process is TMSD-mediated HCR.

In some embodiments, the clusters of amplicons are detected using naked eye, a phase microscope, IRIS, observable sediment formation, SPR, or electric conductivity.

In some embodiments, the methods described herein further comprises labeling the clusters of amplicons prior to the step (c). In some embodiments, the clusters of amplicons are labeled using a DNA binding dye. In some embodiments, the DNA binding dye is an intercalating dye or a groove binding dye. In some embodiments, the DNA binding dye is SYTO-9, SYTO-13, SYTO-82, SYBR Green I, SYBR Gold, EvaGreen, PicoGreen, Ethidium Bromide, Acridine, Propidium Iodide, Crystal Violet, DAPI, 7-AAD, Hoechst, YOYO-1, DiYO-1, TOTO-1, DiTO-1, Hydroxystyryl-Quinolizinium Photoacid, or styryl dye. In some embodiments, the clusters of amplicons are labeled by incorporating a labeled nucleotide. In some embodiments, the clusters of amplicons are labeled using a surface bound or suspended probe. In some embodiments, the surface bound probe is in the same cluster as the targeting nucleic acid. In some embodiments, the surface bound probe is bound by the 3′ end. In some embodiments, the probe is a taqman probe, molecular beacon, or scorpion. In some embodiments, the nucleotide or probe is labeled with biotin, DIG, dppz, Ruthenium(II) complex, gold, Crystal Violet, HRP+TMB, Alkaline phosphatase, palladium, platinum, magnetic nanoparticle, BSA-MnO2 NPs, graphene oxide, Carbon dots, or agents for electrochemical detection. In some embodiments, amplicons of adjacent clusters are labeled differently. In some embodiments, amplicons of different target polynucleotides are labeled the same but at predetermined locations.

In some embodiments, the labeled clusters of amplicons are detected by a scanner, a fluorescence microscope, or a camera, optionally wherein the camera is a cell phone camera. In some embodiments, the number of distinct clusters of amplicons for each target polynucleotide is counted during step (c) to quantify the amount of each target polynucleotide in the sample.

In some aspects, provided herein is a device comprising a first external surface, a second external surface that is different from the first external surface, an internal surface, a plurality of targeting nucleic acids (e.g., primers) immobilized to pre-determined locations on the internal surface, and a reaction chamber enclosed by the internal surface, wherein targeting nucleic acids specific for different target polynucleotides are immobilized at different predetermined locations on the internal surface.

In some embodiments, the device is substantially flat. In some embodiments, the device has a surface/volume ratio larger than 1.3/mm, e.g., larger than 1.4/mm, 1.5/mm, 1.6/mm, 1.7/mm, 1.8/mm, 1.9/mm, 2.0/mm, 3.0/mm, 4.0/mm, 5.0/mm, 6.0/mm, 7.0/mm, 8.0/mm, 9.0/mm, 10/mm, 15/mm, 20/mm, 25/mm, 30/mm, 35/mm, 40/mm, 45/mm, 50/mm, 55/mm, 60/mm, 65/mm, 70/mm, 75/mm, 80/mm, 85/mm, 90/mm, 95/mm, or 100/mm. In some embodiments, the first external surface comprises a material that is able to conduct heat. In some embodiments, the second external surface is on the opposite side to the first external surface. In some embodiments, the second external surface comprise an insulating material. In some embodiments, at least one external surface is transparent. In some embodiments, the device further comprises a strap or a tape to fix the device to a human body.

In some embodiments, the targeting nucleic acid comprises a primer. In some embodiments, only forward primers or reverse primers specific to the target polynucleotides are immobilized to the internal surface. In some embodiments, the reaction chamber comprises the other primer that forms a primer pair with the immobilized forward primer or reverse primer. In some embodiments, both forward and reverse primers specific to the target polynucleotides are immobilized to the internal surface. In some embodiments, the primers comprise a set of nested primers for the same target polynucleotide.

In some embodiments, the targeting nucleic acids specific for each target polynucleotide are uniformly immobilized to the predetermined location of the internal surface. In some embodiments, the plurality of targeting nucleic acids are immobilized as an array of targeting nucleic acid clusters, with each targeting nucleic acid cluster located at one of the predetermined locations on the internal surface. In some embodiments, the targeting nucleic acid clusters are spatially separated from each other in space on the internal surface. In some embodiments, multiple clusters of targeting nucleic acids for the same target polynucleotides are immobilized on the internal surface. In some embodiments, one cluster of targeting nucleic acids spreads into another cluster of targeting nucleic acids within the set of nested targeting nucleic acids for the target polynucleotides. In some embodiments, each cluster of targeting nucleic acids comprises at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or more copies of the same targeting nucleic acids or set of targeting nucleic acids specific for the same target polynucleotide. In some embodiments, the targeting nucleic acid cluster is immobilized to a surface area with a size at least 0.01 mm², 0.0198 mm², 0.02 mm², 0.03 mm², 0.04 mm², 0.05 mm², 0.06 mm², 0.07 mm², 0.08 mm², 0.09 mm², 0.1 mm², 0.2 mm², 0.3 mm², 0.4 mm², 0.5 mm², 0.6 mm², 0.7 mm², 0.8 mm², 0.9 mm², 1 mm², 2 mm², 3 mm², 4 mm², 5 mm², 6 mm², 7 mm², 8 mm², 9 mm², or 10 mm². In some embodiments, the cluster of targeting nucleic acid is in a square shape with a side of 141 micron.

In some embodiments, the targeting nucleic acid is immobilized to the internal surface via 5′ end. In some embodiments, the targeting nucleic acid is covalently linked to the internal surface. In some embodiments, the targeting nucleic acid is 5′ DBCO modified and the internal surface is azide functionalized surface. In some embodiments, the targeting nucleic acid is 5′ amine modified and the internal surface is NHS functionalized surface. In some embodiments, the targeting nucleic acid is non-covalently linked to the internal surface. In some embodiments, the targeting nucleic acid is immobilized to the internal surface via passive absorption, streptavidin-biotin interaction, or hybridization. In some embodiments, the 3′end of the targeting nucleic acid is free to elongate. In some embodiments, the targeting nucleic acids are immobilized on a 2-dimensional surface. In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 10⁴/micron², 5×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, or 5×10⁶/micron³. In some embodiments, the targeting nucleic acid is an invading primer. In some embodiments, the invading primer is LNA, PNA, PTO, ZNA, invader probe, or INA.

In some embodiments, the device further comprises a reaction mix in the reaction chamber. In some embodiments, the reaction mix comprises dNTPs, buffer, and at least one enzyme for isothermal amplification. In some embodiments, the isothermal amplification is TMA, NASBA, LAMP, HIP, HDA, RPA, SDA, or rolling circle amplification.

In some embodiments, the device is used for detecting presence of one or more target polynucleotides in a sample. In some embodiments, the device is used for quantifying the amount of one or more target polynucleotides in a sample. In some embodiments, the device is used at a body temperature. In some embodiments, one or more target polynucleotides are detected using the naked eye or a cell phone camera. In some embodiments, the sample comprises a reference sequence and targeting nucleic acids for the reference sequence are immobilized to the inner surface. In some embodiments, the targeting nucleic acids are positioned in a specific pattern according to a test serial number. In some embodiments, the internal surface is a 3D polymer.

### BRIEF DESCRIPTION OF FIGURES

FIG. 1 is a schematic diagram of a set of spatially separated clusters positioned on a solid surface.

FIGS. 2-6 are schematic representations of certain exemplary embodiments provided herein.

FIG. 7 is an exemplary digital PCR (dPCR) flow chart.

FIG. 8 shows schematic illustration of targeting nucleic acid spotting.

FIGS. 9A-9E show schematic illustration of UMI generation.

FIG. 10 shows schematic illustration of UMI amplification.

FIGS. 11A-11D show schematic illustration of rapid blocking of a cluster using Hinge Initiated Primer dependent amplification (HIP) following first release of UMI from said cluster.

### DETAILED DESCRIPTION

### General

Provided herein are methods and devices related to the detection and/or quantification of one or more target polynucleotides in a sample.

### Definitions

The articles “a” and “an” are used herein to refer to one or to more than one (e.g., to at least one) of the grammatical object of the article. By way of example, “an element” means one element or more than one element.

As used herein, the term “targeting nucleic acid” refers to an immobilized nucleic acid molecule able to specifically bind to a target polynucleotide and/or assist in its amplification, detection, or quantification. In some embodiments, the targeting nucleic acid comprises a primer. In some embodiments, the targeting nucleic acid comprises one or more of a linker, a UMI, a promoter (e.g., T7 promoter), a secondary structure forming stretch (e.g., a 3′ primer blocker, hinge, etc.), a probe, etc. In some embodiments, the targeting nucleic acid has a length that is less than 100 bases, e.g., less than 95, 90, 85, 80, 75, 70, 65, 60, 55, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5 bases.

As used herein, the term “targeting nucleic acid cluster” refers to a spatially separate set of locally immobilized targeting nucleic acids (e.g., primers) designated specifically for (e.g., binding specifically to) a particular target polynucleotide.

The term “binding” or “interacting” refers to an association, which may be a stable association, between two molecules, e.g., between a targeting nucleic acid and target, e.g., due to, for example, electrostatic, hydrophobic, ionic and/or hydrogen-bond interactions.

As used herein, two nucleic acid sequences “complement” one another or are “complementary” to one another if they base pair one another at each position.

As used herein, two or more nucleic acid sequences “correspond” to each other if they are all complementary to the same nucleic acid sequence.

As used herein, “specific binding” refers to the ability of a targeting nucleic acid to bind to a predetermined target. Typically, a targeting nucleic acid specifically binds to its target with an affinity corresponding to a K_{D} of about 10′ M or less, about 10-8 M or less, or about 10-9 M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target.

As used herein, the Tm or melting temperature of two oligonucleotides is the temperature at which 50% of the oligonucleotide/targets are bound and 50% of the oligonucleotide target molecules are not bound. Tm values of two oligonucleotides are oligonucleotide concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

### Methods

In some embodiments, provided herein is a method of quantifying one or more target polynucleotides in a sample, the method comprising: (a) contacting a sample to a solid surface on which a plurality of targeting nucleic acids (e.g., primers) are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) counting the number of distinct clusters of amplicons for each target polynucleotide at the pre-determined location to quantify the amount of each target polynucleotide in the sample.

In some embodiments, provided herein is a method of detecting the presence of one or more target polynucleotides in a sample (if any are present), the method comprising: (a) contacting a sample to a solid surface on which a plurality of targeting nucleic acids are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) detecting the position of clusters of amplicons on the solid surface to detect the presence of one or more target polynucleotides in the sample.

In some embodiments, provided herein is a method for the massive multiplexing of Nucleic Acid Amplification Tests (NAATs), in the sense that there is no limitation of the number of unique primer pairs used in the same reaction compartment, except for the limitation of physical space on the surface. Unique targeting nucleic acids (e.g., pairs of primers) for a specific target polynucleotide are separated in space, positioned in predetermined coordinates, conjugated to the surface, in separate spots. The separation into defined and confined positions prevents cross talk between the pairs of primers, eliminates primer dimers, and precludes the undesired use of one primer as a template to the other, as well as the general forming of a “hairball” of primers that would interrupt the reaction. The usage of predetermined locations enables the use of a uniform label, since the identity of an amplified target is resolved through its specific location and not by the wavelength of a fluorophore (as in suspended qPCR where fluorescence channels form a limitation on the number of different tests one can perform together in the same volume).

In one embodiment, provided herein is a method for digital quantitative parallel oligonucleotide amplification (e.g., digital qPCR, digital RT-qPCR, digital TMA, NASBA, LAMP, HIP, HDA, RPA, SDA, or exponential/linear rolling circle amplification, TMSD-mediated linear/nonlinear HCR, etc.). Each molecule of the marker being quantified can seed its own cluster on the surface; hence the number of distinct clusters thus formed, correlates with the number of molecules bearing this marker in the tested sample. This form of digital quantitative oligonucleotide amplification is cheaper and has a smaller footprint compared to existing methods. This method further allows high throughput, parallel, digital amplification as multiple markers can be handled in this way on a single surface.

In some embodiments, the methods described herein can be used for the detection of any polynucleotide sequence. The method enables parallel detection and quantification of many species of target polynucleotide sequences in a single small reaction volume requiring a very small sample volume.

In some embodiments, the methods described herein can be used for the detection and therefore for diagnostics of pathogen polynucleotide sequences. In some embodiments, the methods described herein enable testing in one run many hypotheses (e.g., different types of viruses, bacteria, cell free DNA, etc.).

In some embodiments, the methods described herein can be used for the detection of known SNPs, insertions, deletions, inversions, translocations, or trisomies (e.g., in cell free DNA) for diagnostics or monitoring in various applications (e.g., planned parenthood, cancer, etc.).

In some embodiments, the methods described herein can be utilized for detection, quantification, and determination of the origin of cell free DNA for diagnostics and monitoring in different applications (e.g., cancer, neurodegenerative disease, liver disease, heart attack, etc.).

In some embodiments, the methods described herein can be used on the bed side or at home for self-diagnosis or telemedicine.

### Sample Preparation

In some embodiments, the one or more target polynucleotides are DNA. In some embodiments, the one or more target polynucleotides are a single-stranded polynucleotides. In some embodiments, the one or more target polynucleotides are double-strand polynucleotides. In some embodiments, method further comprises denaturing any polynucleotides in the sample prior to step (b) to generate single-strand polynucleotides. In some embodiments, a bisulfate conversion of the DNA sample occurred before step (a).

In some embodiments, the target polynucleotides are RNA. In some embodiments, reverse transcriptase is contacted to the sample before step (b).

In some embodiments, the sample is diluted prior to the step (a).

In some embodiments, the methods described herein further comprise a sample preparation step, for example, a nucleic acid purification step, a sample enrichment step, or a reverse transcription step for RNA target molecule. In some embodiments, the sample preparation step (e.g., sample enrichment or purification, reverse transcription, etc.) can be done directly on the solid surface or within the device described herein.

In some embodiments, the sample is a purified nucleic acid (e.g., a purified DNA sample, a purified RNA sample, etc.) sample. In some embodiments, the sample is an unpurified sample, and the methods described herein include a nucleic acid purification step prior to contacting the sample to a solid surface on which a plurality of targeting nucleic acids are immobilized. The sample can be purified using any known methods in the art for DNA or RNA purification, or using commercially available kits.

In some embodiments, the sample is an unpurified sample, and the surface-bound targeting nucleic acids (e.g., primers) are used to simplify sample purification. As the targeting nucleic acids are conjugated to a solid surface, they can be used as capturing probes in the sense that after incubation with a sample, the surface can be washed to remove all unbound material including unrelated oligos, nucleases, proteases, and other contaminations or potential reaction inhibitors, leaving behind mostly the target nucleic-acid molecules hybridized to the surface-immobilized targeting nucleic acids. After the wash, the reaction can go straight ahead on the solid surface. This process could be done repeatedly with more and more sample, thus enriching the target oligos on the surface. In case of RNA target, at this point a reverse transcriptase can be introduced to elongate the binding primers into the wanted cDNA.

In some embodiments, the step (a) comprises (i) annealing the targeting nucleic acid to the corresponding target polynucleotide in the sample, (ii) washing the solid surface to remove contaminates from the sample, (iii) contacting the solid surface with a reaction mix.

In some embodiments, the methods described herein can be used to detect and/or quantify one or more target polynucleotides in a pool of samples. For example, in some embodiments, the oligo sample can be extended with a specific oligonucleotide barcode, either from one end of the strand or from both ends. Following that, pooled samples can be applied to the solid surface on which a plurality of targeting nucleic acids (e.g., primers, or primers and probes) are immobilized. In some embodiments, the barcode is attached to one end of the sample oligo, and spots hold one primer to complement and elongate the barcode side and one primer to complement the target oligo (to be detected) side. To increase specificity, spots can hold probes specific to the target. In some embodiments, the barcode is attached to both ends of the sample oligo, and spots hold primers complementing the barcodes fitting to amplify the stretch held between them, and probes specific to the target. In some embodiments, for each sample, some spots are designed to amplify the stretch between the 5′ end barcode and a 3′ end target marker, some designed to amplify the stretch between a 5′ end target marker and a 3′ end barcode and some to amplify the stretch between the 2 barcodes. Probes specific for the target can be added to all types of spots. In some embodiments, the barcode attachment is done while an initial signal amplification is done.

### Immobilized Targeting Nucleic Acid Clusters

In some embodiments, the immobilized targeting nucleic acid clusters comprise immobilized primers. In some embodiments, only forward primers or reverse primers specific to the target polynucleotide are immobilized to a solid surface. In some embodiments, when only one primer from a pair is immobilized to the solid surface, the other primer that forms a primer pair with the immobilized forward or reverse primer is contacted to the solid surface prior to amplification step (b). In some embodiments, the other primer that forms a primer pair with the immobilized primer is generated at step (b) (e.g., in an SDA process). In some embodiments, both forward and reverse primers specific to the one or more target polynucleotides are immobilized to the solid surface. In some embodiments, primers can be found both immobilized in separate clusters and in suspension. In some embodiments, suspension amplification uses general mode of amplification (e.g., using degenerate primers, or conserved/similar flanking regions) while solid-phase amplification is specific (e.g., using nested primers, specific primers, etc.). In some embodiments, both forward and reverse primers from a pair of primers that are specific for a target polynucleotide are generated at step (b).

In some embodiments, the primers are uniformly immobilized to the predetermined location of the solid surface. In some embodiments, when primers are uniformly attached to a predetermined area, the amplification process is stopped before clusters merge into each other in order to count.

In some embodiments, the primers are immobilized to the solid surface as an array of primer clusters, with each primer cluster located at one of the predetermined locations on the solid surface. In some embodiments, the primer clusters are spatially separated from each other on the solid surface. Preferably, primers are conjugated densely enough to allow the intended amplicon growing from one primer, anneal to the other primer, and allowing the polymerization of its complement. In some embodiments, the targeting nucleic acids are immobilized on a 2-dimensional surface. In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 4/micron², 5×10⁴/micron², 6×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, 5×10⁶/micron³, or 8×10⁶/micron³.

A spotted array is preferable to the uniformly covered surface in order to keep clusters confined to a defined area and avoid the merge of clusters, especially for continuous amplification. A spotted and aligned array can be preferable to allow for a more straightforward data analysis as clusters' pre-determined position and the total number of clusters is known. It can simplify data-analysis to decide whether a position is populated or not, and what is the fraction of populated positions.

In some embodiments, at least about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹ or 10¹⁰ distinct primers are immobilized onto the surface. In some embodiments, each primer cluster comprises at least about 11, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or more identical primer molecules.

In some embodiments, different clusters can hold: (1) same detection agent (e.g., primers) for robustness or for target counting (signal from more clusters indicates more targets found); (2) different detection agent for the same target for increased robustness and positive predictive value; or (3) different detection agents for different targets for parallel detections of different targets.

In some embodiments, multiple different targeting nucleic acids for detecting the same target polynucleotides are immobilized on the solid surface. In some embodiments, multiple different targeting nucleic acids for detecting more than one target polynucleotides are immobilized on the solid surface. These target polynucleotides can be from the same cell, organism, tissue, patient, or pathogen. In some embodiments, the method further comprises a step of using logical “and”/“or” gates of more than one target polynucleotides to increase specificity and sensitivity.

In some embodiments, the methods described herein can be used to test multiple markers to increase sensitivity and specificity. In order to account for the low specificity that may occur (e.g., due to the low annealing temperatures, etc.), multiple markers can be used in combination as a logical “and” gate, or multiple clusters indicating the same marker is found multiple times. In some embodiments, to increase sensitivity, multiple markers may be used in combination as a logical “or” gate. In some embodiments, the primers comprise a set of nested primers for the same target polynucleotide. In some embodiments, one cluster of primers spreads into another cluster of primers within the set of nested primers for the target polynucleotides. In some embodiments, some adjacent clusters can hold nesting primers in such a way that only if the right target oligo is amplified, the amplification can continue to the next cluster. As in regular nesting PCR, this decreases false positive cases, because the target oligo can hold both sets of primers, whereas there is a low chance that a different oligo may hold both sets of primers.

In some embodiments, during the amplification process, amplified signal is released to the suspension and reseeds a different cluster (e.g., when TMA, NASBA, or SDA is used for the amplification step), the amplified signal can be different from the amplification initiating target.

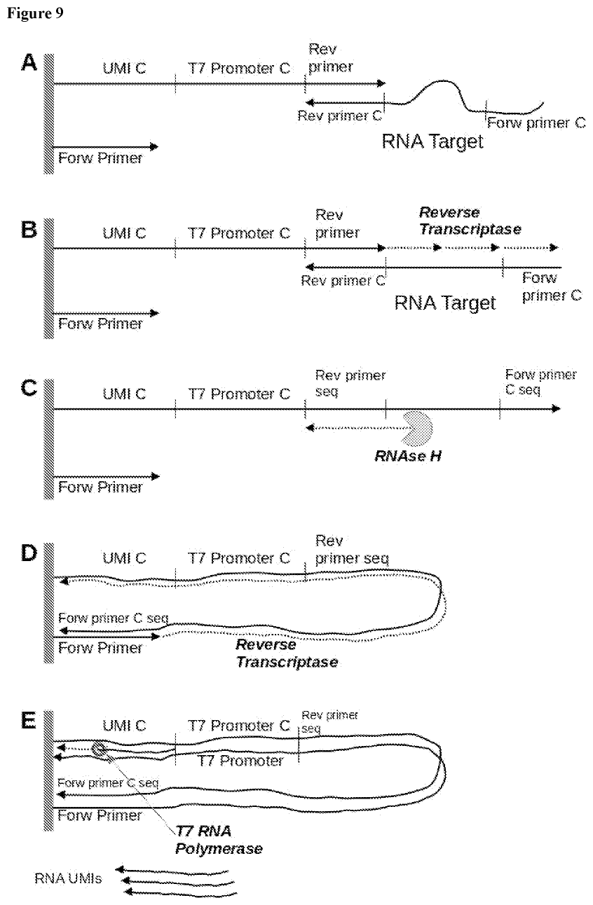

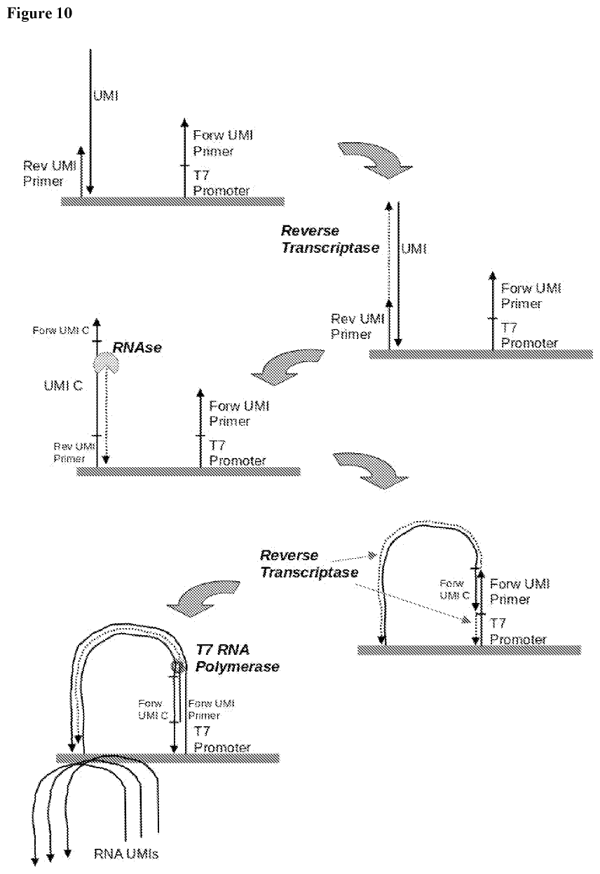

In some embodiments, a surrogate signal, different from the amplification initiating target, is amplified to generate the detectable signal. For example, in some embodiments, NASBA/TMA amplification method is used, and the signal RNA, as in a regular reaction, anneals to the reverse primer (FIG. 9A). The reverse primer is then elongated by the reverse transcriptase, according to the RNA target template (FIG. 9B). After the elongation, the RNA target, in the double strand RNA-DNA, is degraded by the RNAse H activity (FIG. 9C). The newly formed DNA reverse complement target anneals to the forward primer. The forward primer, in turn, elongates, and a dsDNA forms (FIG. 9D). One of the primers, in these embodiments, is designed to hold on its 5′ end a unique molecular identifier (UMI), and then a T7 promoter complementary stretch upstream to the primer. In these embodiments, the dsDNA holds the T7 promoter upstream to the UMI. Then the T7 polymerase generates copies of the UMI to be released to the reaction suspension (FIG. 9E). These UMI RNA copies find designated primer clusters for each specific UMI (FIG. 10). In some embodiments, SDA method is used for amplification, and the nick is designed to form on the target side. The primer is designed such that the UMI is at the 5′ tail. The target complementary to the primer is nicked and elongated according to the UMI template. In some embodiments, each UMI cluster type is generated of a single target molecule and as such, each UMI cluster formed can be count as 1 original target found in the sample. In some embodiments, each UMI cluster type is generated of at list a single target molecule and as such, each UMI cluster formed can be count as, at list, 1 original target found in the sample (e.g. 1 or more original targets). In some embodiments, the transition from original target to UMI is done in suspension.

In some embodiments, after the first target oligo seeds a UMI cluster, the option for a second target oligo to reseed the UMI type transition cluster is prevented. In some embodiments, the mechanism to achieve said prevention is by initiating local amplification (e.g., HIP), as to rapidly populate the cluster.

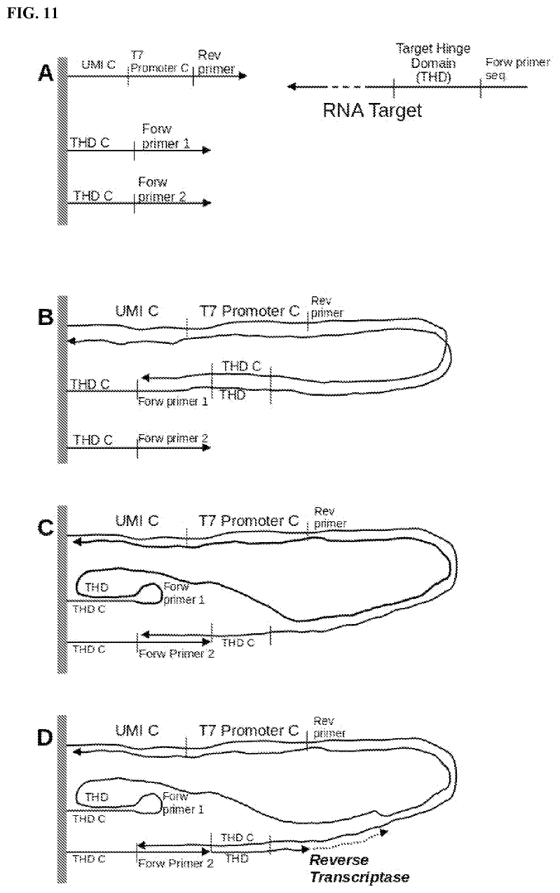

The forward primer has an additional domain at the 5′ end, which is complementary to a sequence of bases which are part of the RNA Target sequence, located upstream to the target's reverse primer complementary domain (that is, closer to the 5′ of the RNA target sequence), which is identified below as the Target Hinge Domain complementary (THD C) (FIG. 11A).

In some embodiments the THD C is an invading stretch (e.g. made in full or in part with PNA, LNA, etc.).

A dsDNA is formed as described above, i.e., reverse transcriptase elongates the reverse primer, RNAse H degrades the target RNA strand, the forward primer binds the 3′ end of the strand extending the reverse primer, and reverse transcriptase elongates the forward primer (FIG. 11B).

The complementary sequence thus constructed, extending forward primer 1, contains both a THD and a THD-complementary domain, separated by the forward primer sequence. Additional forward primers are immobilized on the surface, and an energetically favorable configuration is for the THD-complementary domain of the forward primer 1 strand to pair with the THD on the same strand, while a distinct forward primer 2 molecule hybridizes with the forward primer complementary sequence on the 3′-end of the reverse complement target strand (FIG. 11C).

Reverse transcriptase can then elongate forward primer 2 against the reverse complement target strand, thereby separating the forward strand extended from forward primer 1 from the reverse complement strand. After elongation, the forward primer 2 strand has both a THD domain and a THD-complementary domain, so that the process can continue with yet another forward primer (FIG. 11D).

The forward primer 1 strand, which is separated from the reverse primer strand by the reverse transcriptase, can anneal to a different reverse primer immobilized on the surface. Thus an exponential reaction takes place, eventually blocking all the reverse primers within the confines of the cluster, and preventing additional targets from hybridizing into a cluster whose UMI has already been triggered.

In some embodiments, a set of primer clusters are designed, each being the nested set of primers of the specific stretch of oligo screened for. In some embodiments, during the amplification process, amplified signal is not released to the suspension, and the nested primer cluster is concatenated in a way that one can spread in to the other.

In some embodiments, the sample comprises a reference sequence, and primers for the reference sequence are immobilized to the solid surface.

In some embodiments, the targeting nucleic acids are immobilized to the solid surface via its 5′ end. In some embodiments, the targeting nucleic acids are immobilized to the solid surface via either 5′ or 3′ end, and a non-enzymatic amplification process is used at the step (b). In some embodiments, the targeting nucleic acids are covalently linked to the solid surface. In some embodiments, the user can change the analyzed target (i.e., customize the markers) between runs so that the methods described herein can be used in a more flexible way. One exemplary method that can be employed to select and cover the surface with the desired targeting nucleic acids is the use of click chemistry-covered surfaces functionalized with azide, NHS, etc. Targeting nucleic acids are designed with the appropriate modification on the 5′ end (e.g., DBCO for azide functionalized surfaces, or amine for NHS functionalized surfaces, etc.). The first step in such an exemplary method is applying the 5′ modified targeting nucleic acids on the functionalized surface to allow for the conjugation of the targeting nucleic acids to the surface. The surface is then washed thoroughly to get rid of any unbound targeting nucleic acid, and all of the unused functional groups are blocked for any non-specific binding. Additional methods for covalently linking the targeting nucleic acids to the solid surface include but are not limited to, e.g., azide-alkyne; Amine-reactive —NH2+NHS ester/Imidoester/Pentafluorophenyl ester/Hydroxymethyl phosphine/Epoxide/Isocyanate; Carboxyl-to-amine reactive —COOH+Carbodiimide (e.g., EDC); Sulfhydryl-reactive —SH+Maleimide/Haloacetyl (bromo-, chloro-, or iodo-)/Pyridyl disulfide/Thiosulfonate/Vinyl sulfone; Aldehyde-reactive (e.g., oxidized sugars, carbonyls) —CHO+Hydrazide/Alkoxyamine/NHS ester; Hydroxyl (nonaqueous)-reactive —OH+Isocyanate; photoreactive cross linking(e.g., aryl azides+nucleophile (e.g., primary amine), diazirine+amino acid side chain or peptide backbone), etc.. In some embodiments, the targeting nucleic acids are 5′ DBCO modified and the solid surface is azide functionalized surface. In some embodiments, the targeting nucleic acids are 5′ amine modified and the solid surface is NHS functionalized surface.

A different exemplary method for covering the surface with the user primers is elongating the already attached primers, populating the surface according to a template of the user design. This template can be designed such that its 3′ stretch complements the primers already immobilized to the solid surface and the 5′ stretch complements the intended primer.

In this exemplary method, the first step is inserting a high concentration of both of the user-designed DNA strands, annealing to the already existing, surface-attached, primers, and elongating using DNA polymerase to generate the desired primers. All templates are then denatured and washed thoroughly off the reaction compartment.

In some embodiments, wherein steps of denaturation in aggressive heat or chemical reagent are not used, the targeting nucleic acids are non-covalently linked to the solid surface, e.g., via passive absorption, streptavidin-biotin, hybridization, etc.

In some embodiments, the 3′end of the targeting nucleic acids are free to elongate.

In some embodiments, the targeting nucleic acids are invading primers, e.g., LNA, PNA, PTO, ZNA, invader probe, or INA.

In certain embodiments the surface can be any solid support. In some embodiments, the surface is the surface of a flow cell. In some embodiments, the surface is a slide, a chip (e.g., the surface of a gene chip), a microwell plate, a plate, a tube, or a fluidic channel. In some embodiments, the surface is a bead (e.g., a paramagnetic bead). Cartridges formats can be varied, for example, a well plate (e.g. 96 well plate) can be used to digitally test different samples or different markers in every well. A different example is the use of fluidics channels, which can be integrated into a machine that allows for washing steps.

In some embodiments, the surface is an inner surface of the devices described herein. In some embodiments, the surface is a 3D polymer.

### Amplification

In some embodiments, the amplification process is a semi-solid phase amplification with one end on the solid surface and the other end in suspension. In some embodiments, the amplification process is a solid phase amplification. In some embodiments, amplification can occur both in suspension and in solid phase at the same time.

In some embodiments, the solid phase amplification process is a bridge amplification. The amplification process can be a stepwise thermal or chemical amplification, e.g., PCR, RT-PCR, etc. In some embodiments, the amplification process is an isothermal amplification, including but not limited to, e.g., TMA, NASBA, LAMP, HIP, HDA, RPA, SDA, or exponential/linear rolling circle amplification, etc.. Isothermal amplification methods, such as HIP, LAMP, HDA, RPA, SDA, NASBA, TMA, etc., are known in the art. For example, HIP is described in Fischbach et al. (2017) Scientific Reports, 7: 7683| DOI:10.1038/s41598-017-08067-x; low temperature LAMP is described in Cai et al. (2018) Anal. Chem. 90:8290-8294; HDA is described in Vincent et al. (2004) EMBO Rep. 5:795-800; TMA is described in Brentano and Mcdonough (2000) Nonradioactive Analysis of Biomolecules 374-380; RPA is described in Piepenburg O et al. (2006) PLOS Biology 4(7): e204.; SDA is described in Walker G T et al. (1992) Nucleic Acids Research 7:1691-1696; NASBA is described in Deiman B. et al. (2002) Molecular Biotechnology 20:163-179, each of which is incorporated by reference herein in its entirety. In some embodiments, the amplification is non-enzymatic amplification process, e.g., TMSD-mediated linear/nonlinear HCR, etc.

In some embodiments, the amplification process occurs at an ambient temperature. In some embodiments, the amplification process occurs at about 37° C. to about 42° C. In some embodiments, the amplification process occurs at human body temperature. In some embodiments, the amplification process occurs without a mechanical heating device. In some embodiments, heat induced by a chemical exothermic reaction for the amplification process.

Different primer configurations may be used for different amplification methods. For example, in some embodiments, stepwise thermal or chemical PCR amplification method is used, both of the primers specific to the intended target would be densely attached to the surface. The surface can be either spotted with an array of primer spots or be uniformly covered with the primers. In some embodiments, a chemical PCR process is used, and the sample is applied in a single strand form (for example, by using the Illumina sample denaturation and dilution protocol). In some embodiments, a thermal PCR process is used, and the first step is denaturation of double stranded DNA sample to generate single strand form. Alternatively, invading primers (e.g., LNA, PNA, PTO, ZNA, Invader probes, INA, etc.) can be used.

In some embodiments, annealing of the target polynucleotides to the primers can then take place. Incubation temperature and time should be adjusted according to the sample complexity, the amplification method, and the primers Tₘ. These parameters can impact the sensitivity and the specificity of the reaction. In some embodiments, the polymerase elongates the primers attached to a target after the annealing step. The annealing and elongation steps can be considered as the seeding step. In some embodiments, denaturation and washing steps can be applied to ensure that only covalently bound material stays in the system and ends the seeding step. In some embodiments, the washing step is not applied and continuous seeding occurs. If the washing step is done, new amplification reagent (e.g., PCR reagent) should be introduced to the reaction compartment. In some embodiments, rounds of bridge amplification (e.g., bPCR) can generate clusters of the amplified target. Each cluster is seeded by (i.e., originated out of) a single target molecule seed. The number of clusters correlates with the number of target molecules found in the sample applied to the surface. The number of rounds depends on the size of cluster needed for detection according to the sensitivity of the detection method used. In certain embodiments, probes are used in the detection method, and extra steps allowing for specific probe annealing take place.

In some embodiments, the starting material is RNA, and no denaturation takes place in sample preparation and the elongation step in the seeding stage is done using reverse transcriptase.

In some embodiments, RPA isothermal amplification is used. In this case, the denaturation step is not relevant, washing steps are not necessary, and amplification does not require a heating device as the reaction can occur in an ambient temperature. This allows implementing the amplification process in a simple device, such as the devices described herein.

In some embodiments, TMA or NASBA isothermal amplification is used. In this case, the starting material is single stranded DNA or RNA (dsDNA is also possible if invading primers are used), and a low temperature, for example, 37-42° C., can be used. In some embodiments, human body temperature should suffice, and no heating device is needed.

### Labeling and Detection

In some embodiments, the methods described herein comprise labeling the clusters of amplicons prior to the detection step. In some embodiments, the clusters of amplicons are labeled using DNA binding dyes such as intercalating dye, groove binding dyes, etc., including but not limited to, e.g., SYTO-9, SYTO-13, SYTO-82, SYBR Green I, SYBR Gold, EvaGreen, PicoGreen, Ethidium Bromide, Acridine, Propidium Iodide, Crystal Violet, DAPI, 7-AAD, Hoechst, YOYO-1, DiYO-1, TOTO-1, DiTO-1, Hydroxystyryl-Quinolizinium Photoacid, or styryl dye.

In some embodiments, the clusters of amplicons are labeled by incorporation of an intrinsically fluorescent or labeled nucleotide, e.g., an intrinsically fluorescent or labeled dUTP, dGTP, dCTP, dATP, etc.

In some embodiments, the clusters of amplicons are labeled by a surface bound or suspended probe, e.g., a Taqman probe, molecular beacon, or scorpion, etc. In some embodiments, the surface bound probe is in the same cluster as the primer. In some embodiments, the surface bound probe is bound by the 3′ end. In some embodiments, the amplified amplicons are released to the suspension, and the detecting probes can be in separate clusters.

The probes, labeled dNTPs, and groove binding agents can be labeled with many different molecules for either immediate or for secondary reporting. For example, they can be labeled with biotin, DIG, and with either fluorescent, luminescent (e.g., dppz, Ruthenium (II) complex, etc.), colorimetric (e.g., gold, Crystal Violet, HRP+TMB, Alkaline phosphatase, palladium, platinum, magnetic nanoparticle, BSA-MnO2 NPs, graphene oxide, Carbon dots), or electrochemical detection.

In some embodiments, the clusters of amplicons are not labeled, and the detection is based on the change in physical qualities occurring due to the amplification on a specific spot e.g., change in the optical properties (e.g. refraction, opacity, etc.) (can be detected using phase microscopy, IRIS, etc.), observable sediment formation, SPR, electric conductivity, etc.

In some embodiments, amplicons of different target polynucleotides are labeled differently. In some embodiments, adjacent clusters are labeled differently. In some embodiments, amplicons of different target polynucleotides are labeled the same but at pre-determined locations.

### Devices

In some aspects, provided herein is a device comprising a first external surface, a second external surface that is different from the first external surface, an internal surface, a plurality of targeting nucleic acids (e.g., primers) immobilized to pre-determined locations on the internal surface, and a reaction chamber enclosed by the internal surface, wherein targeting nucleic acids specific for different target polynucleotides are immobilized at different predetermined locations on the internal surface.

In some embodiments, the device is substantially flat. In some embodiments, the device has a surface/volume ratio larger than 1.3/mm, e.g., larger than 1.4/mm, 1.5/mm, 1.6/mm, 1.7/mm, 1.8/mm, 1.9/mm, 2.0/mm, 3.0/mm, 4.0/mm, 5.0/mm, 6.0/mm, 7.0/mm, 8.0/mm, 9.0/mm, 10/mm, 15/mm, 20/mm, 25/mm, 30/mm, 35/mm, 40/mm, 45/mm, 50/mm, 55/mm, 60/mm, 65/mm, 70/mm, 75/mm, 80/mm, 85/mm, 90/mm, 95/mm, or 100/mm.

In some embodiments, the first external surface comprises a material that is able to conduct heat. For example, the material for the first external surface can be selected from thermally conductive material known in the art, for example, a metal (e.g., copper, brass, aluminum, a metal alloy), beryllium oxide ceramics, or a thermally conductive plastic/polymer. Preferably, the external surface of the device that is closest to the internal surface to which the targeting nucleic acids are attached can have high heat conductivity to allow for the transfer of body heat to the reaction chamber. This surface can be placed so that it is in direct contact with the body. In some embodiments, there may be thermally conductive gap-filler or adhesives between the first external surface and the reaction chamber.

In some embodiments, the second external surface is on the opposite side to the first external surface. In some embodiments, the second external surface comprises an insulating material. The insulating material can minimize the heat loss to the surrounding environment. In some embodiments, thermal insulation of the chamber may be achieved either inside the device between the reaction chamber and the second external surface, and/or by the second external surface being composed of a thermally insulating material. Internally, the insulator may be an air gap, or a space filling material such as corrugated paper, or heat insulating foams. The second external surface itself may be composed of a material of low thermal conductivity, such as polypropylene or polyethylene.

In some embodiments, at least one external surface is transparent to provide a view of the reaction progress. In some embodiments, all external surfaces are transparent.

In some embodiments, the device further comprises a strap or a tape to fix the device to a human body.

In some embodiments, the targeting nucleic acids are immobilized to the inner surface of the device using methods and/or in manners described herein, for example, using methods and/or in manners described above for immobilizing targeting nucleic acids to a solid surface.

In some embodiments, the device further comprises a reaction mix in the reaction chamber. In some embodiments, the reaction mix comprises dNTPs, buffer, and at least one enzyme for isothermal amplification. In some embodiments, the isothermal amplification is TMA, NASBA, LAMP, HIP, HDA, RPA, SDA, or rolling circle amplification.

In some embodiments, the device is used for detecting presence of one or more target polynucleotides in a sample. In some embodiments, the device is used for quantifying the amount of one or more target polynucleotides in a sample.

In some embodiments, the device is used at a body temperature.

In some embodiments, one or more target polynucleotides are detected using the naked eye or a cell phone camera.

In some embodiments, the sample comprises a reference sequence and targeting nucleic acids for the reference sequence are immobilized to the inner surface.

In some embodiments, the targeting nucleic acids are positioned in a specific pattern according to a test serial number.

### Examples

### Example 1—Signal Amplification Using Bridge PCR

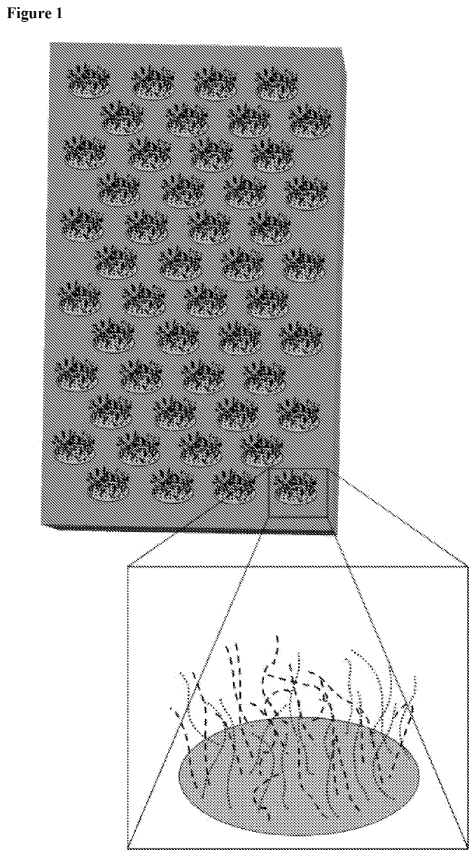

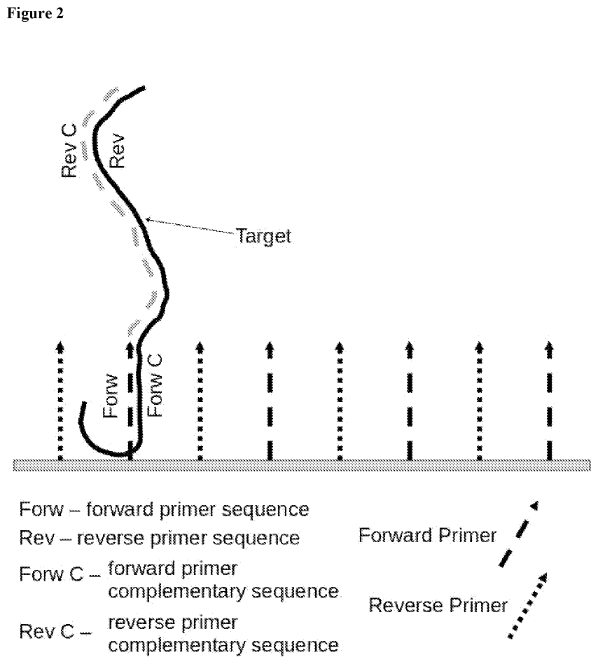

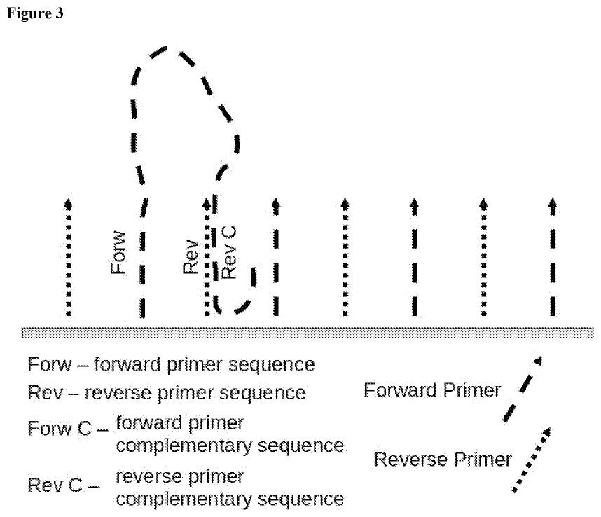

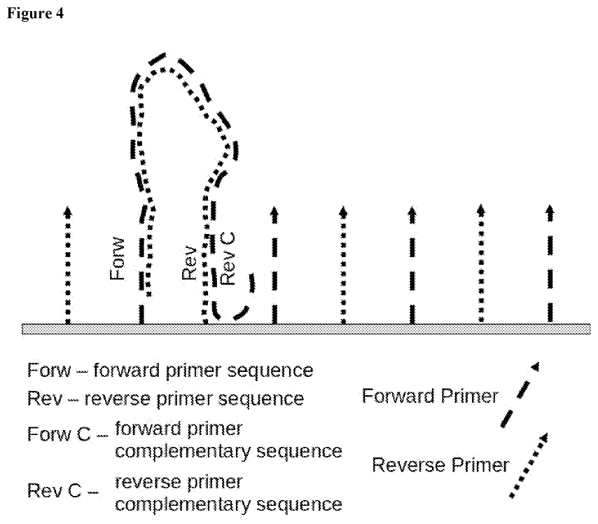

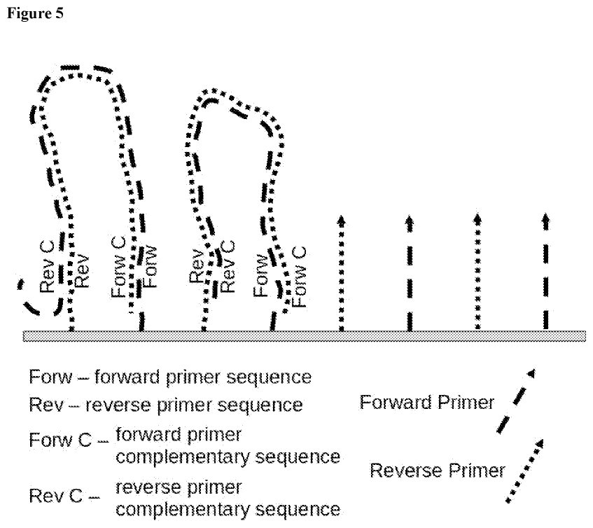

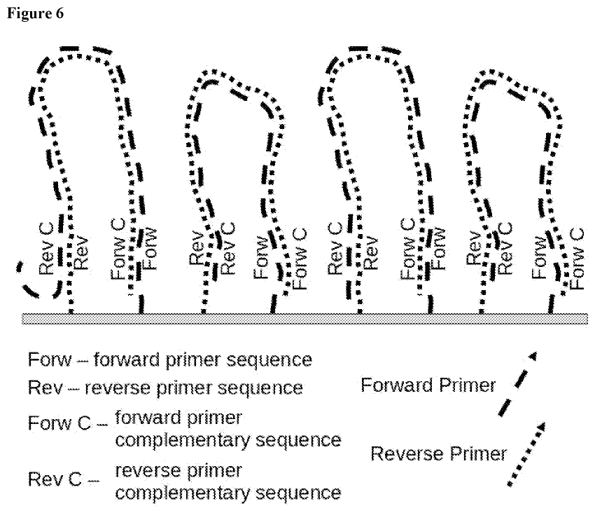

In some embodiments, the PCR process can be either an isothermal PCR or thermal steps-based PCR. In some embodiments, a set of spatially separated clusters, are positioned on a solid surface (FIG. 1). Each cluster can hold both the forward and reverse primers specific for one or more oligo targets. The dense primers in a cluster can create high local concentration of the specific primers. As seen in FIGS. 2 and 3, the target can find one of its corresponding primers and initiates a Polymerization reaction, generating the complement for the reverse primer found in the same cluster. As seen in FIGS. 4-6, after the displacement of the target the bridge PCR reaction is initiated, generating a cluster in the predefined location on the surface. At the end of the run, which of the clusters a PCR reaction occurred is detected, and based on this information the targets are identified and quantified. In one embodiment, each square of surface holds many copies of two unique primers, specific for one molecular marker-oligo target. If an oligo target appears in the sample, a cluster appears on the appropriate square, and each cluster is an amplification of exactly one oligo strand. In some embodiments, each square surface holds an array of primer clusters that may be homogeneous, and specific to one oligo target.

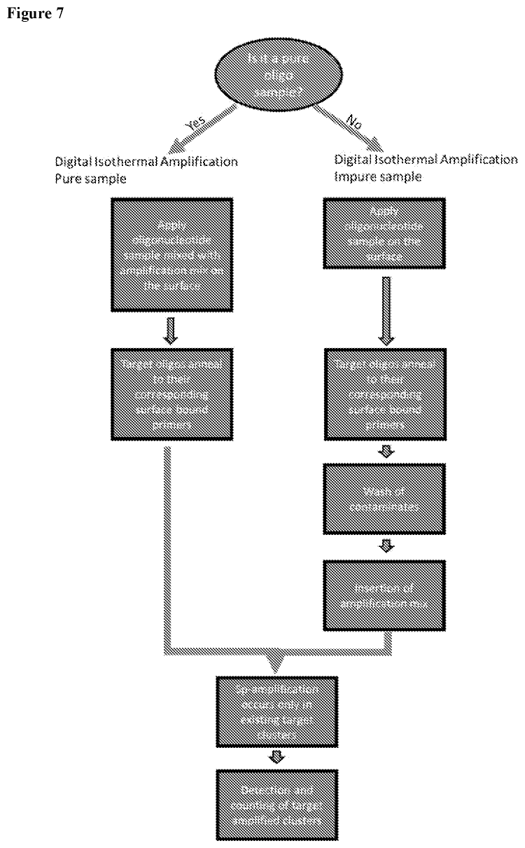

FIG. 7 shows an exemplary digital PCR (dPCR) flow chart.

### Example 2—TMA

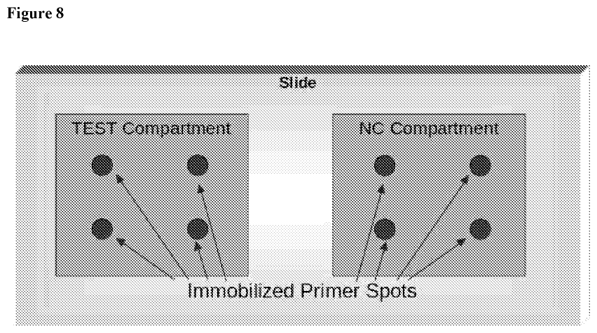

A 3D azide glass slide was used as the solid surface. 5′ DBCO modified reverse primers, were spotted in duplicates on the glass slide. The spotting pattern was repeated in 2 locations on the slide (FIG. 8). The glass with the spots was incubated overnight at RT (room-temperature) and then washed 3 times with 1.5×SSC, 0.1% SDS and 1 time with UPW.

The spotted glass was then blocked using 0.1% BSA and 0.2 mg/ml salmon sperm for 30 min at RT and then washed with UPW and dried. An NASBA mix was prepared using the amsbio NASBA wet kit according to the manufacturer protocol. Only forward primer was added to the mix. A labeled dUTP was added to the prepared mix. Before adding the enzyme cocktail to the NASBA mix, the mix was split to two. The enzyme cocktail was added to one and the other was supplemented with UPW instead to serve as the negative control. The full mix was applied on one of the spotting locations and the NC (negative control) mix was applied on the other spotting location. Both locations were then covered by cover slips and sealed using Photo glue. When the photo glue dried off, the slide was incubated in 41° C. for 90 min. After incubation, the glue and the coverslips were removed and the glass was washed 4 times using 1.5×SSC, 0.1% SDS. Then the glass was incubated for 15 minutes with two different probes—cy5 reverse primer probe and fam reverse complement target probe. The latter was composed of LNA bases in order to facilitate strand invasion. Probe mix was supplemented with 0.2 mg/ml salmon sperm to decrease nonspecific binding of the probes. After the incubation, washing was repeated and the glass was inspected using a fluorescence microscope. The microscope image of the full NASBA reaction demonstrated collocated fluorescence in the three channels that were imaged—Cy5, labeled dUTP, and FAM. The cy5 channel confirms that the reverse primers were successfully immobilized to the surface, and indicates the location of the spot. The collocated fluorescence in the labeled dUTP channel indicates that a polymerization of a nucleic acid strand was accomplished. The collocated fluorescence in the FAM channel confirms that the probe for the reverse target hybridizes with the said strand as expected. In contrast, the microscope image for the negative control spots, where the enzyme was missing, demonstrated fluorescence only in the cy5 channel, indicating that no polymerization took place in that compartment.

### Example 3—TMA

In a follow-up experiment, the two sets (TEST/NC) of spots held a mixture of both forward and reverse primers. No primers were in the solution mix. As described for the previous experiment, the mix was split in two: the full reaction (TEST) and no enzyme (NC). Imaging the three fluorescent channels in a microscope, the spot with both primers immobilized and with the NASBA enzyme demonstrated all three collocated channels, indicating successful amplification of the signal. The spot with both primers but without NASBA enzyme only showed a fluorescent channel in the cy5 channel, indicating the existence of the reverse primers, but no polymerization taking place.

### Example 4—PCR

In a similar fashion, spots of either reverse or forward primer were prepared. A PCR mix, with no primers, supplemented with labeled dUTPs, was prepared and applied on the conjugated primer spots. Single stranded DNA target was added in excess to the mix. The reaction was compartmentalized and sealed using BioRad frame seal for in situ PCR. The slide was loaded into a thermocycler using a “96 wells to slides” adapter for 1 round of polymerization. The frame seal was then removed and the slide washed, incubated with probes, washed again and results were inspected under a fluorescence microscope as described before for the NASBA reaction. Specific polymerization using 1 round PCR reaction on slide was done.

Primers were only in the spots covalently attached to the glass slide. Large excess of ssDNA target was used. Inspection of the spot where the reverse primer was immobilized demonstrated a collocated signal for the three fluorescent channels, indicating successful polymerization of the reverse target in this spot. In contrast, when inspecting the spot in which the forward primer was immobilized, an irrelevant primer in these settings, no significant signal was visible, indicating no polymerization occurred.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of quantifying one or more target polynucleotides in a sample, the method comprising:
(a) contacting a sample to a solid surface on which a plurality of targeting nucleic acids are immobilized, wherein targeting nucleic acids specific for different target polynucleotides are immobilized at different predetermined locations on the solid surface;
(b) performing an amplification process on the solid surface such that the presence of a target polynucleotide in the sample results in the generation of a cluster of immobilized amplicons on the solid surface at the predetermined location at which the targeting nucleic acids specific for that target polynucleotide was immobilized; and
(c) counting the number of distinct clusters of amplicons for each target polynucleotide at the pre-determined location to quantify the amount of each target polynucleotide in the sample.

2. A method of detecting the presence of one or more target polynucleotides in a sample, the method comprising:
(a) contacting a sample to a solid surface on which a plurality of targeting nucleic acids are immobilized, wherein targeting nucleic acids specific for different target polynucleotides are immobilized at different predetermined locations on the solid surface;
(b) performing an amplification process on the solid surface such that the presence of a target polynucleotide in the sample results in the generation of a cluster of immobilized amplicons on the solid surface at the predetermined location at which the targeting nucleic acids specific for that target polynucleotide was immobilized; and
(c) detecting the position of clusters of amplicons on the solid surface to detect the presence of one or more target polynucleotides in the sample.

3. The method of claim 1 or 2, wherein the targeting nucleic acids comprise primers.

4. The method of claim any one of claims 1-3, wherein primers are generated during step (b).

5. The method of claim 4, wherein the primers are generated during step (b) via strand displacement amplification (SDA).

6. The method of any one of claims 3-5, wherein only forward primers or reverse primers specific to the target polynucleotide are immobilized to the solid surface.

7. The method of claim 6, wherein the other primers that form a primer pair with the immobilized forward primers or reverse primers are contacted to the solid surface prior to step (b).

8. The method of claim 1-7, wherein both forward and reverse primers specific to the one or more target polynucleotides are immobilized to the solid surface.

9. The method of any one of claims 1-8, wherein the targeting nucleic acids are uniformly immobilized to the predetermined location of the solid surface.

10. The method of any one of claims 1-8, wherein the targeting nucleic acids are immobilized to the solid surface as an array of targeting nucleic acid clusters, with each targeting nucleic acid cluster located at one of the predetermined locations on the solid surface.

11. The method of claim 10, wherein the targeting nucleic acid clusters are spatially separated from each other on the solid surface.

12. The method of claim 10 or 11, wherein multiple clusters of targeting nucleic acids for the same target polynucleotides are immobilized on the solid surface.

13. The method of claim 12, wherein the targeting nucleic acids comprise a set of nested primers for the same target polynucleotide.

14. The method of claim 13, wherein one cluster of primers spreads into another cluster of primers within the set of nested primers for the target polynucleotides.

15. The method of claim 10 or 11, wherein multiple clusters of targeting nucleic acids for detecting more than one target polynucleotides from the same cell, organism, or pathogen are immobilized to the solid surface.

16. The method of any one of claims 9-15, wherein each cluster of targeting nucleic acids comprises at least 2 copies of the same targeting nucleic acids.

17. The method of any one of claims 1-16, wherein the targeting nucleic acids are immobilized to the solid surface via its 5′ end.

18. The method of any one of claims 1-17, wherein the targeting nucleic acids are covalently linked to the solid surface.

19. The method of claim 18, wherein the targeting nucleic acids are 5′ DBCO modified and the solid surface is azide functionalized surface.

20. The method of claim 18, wherein the targeting nucleic acids are 5′ amine modified and the solid surface is NHS functionalized surface.

21. The method of any one of claims 1-17, wherein the targeting nucleic acids are non-covalently linked to the solid surface.

22. The method of claim 21, wherein the targeting nucleic acids are immobilized to the solid surface via passive absorption, streptavidin-biotin interaction, or hybridization.

23. The method of any one of claim 1-22, wherein the 3′end of the targeting nucleic acids are free to elongate.

24. The method of any one of claim 1-23, wherein each cluster comprises targeting nucleic acids at a density of at least 9/micron².

25. The method of any one of claims 1-24, wherein the targeting nucleic acids are invading primers.

26. The method of claim 25, wherein the invading primers are LNA, PNA, PTO, ZNA, invader probe, or INA.

27. The method of any one of claim 1-26, wherein the solid surface is a slide, a chip, a microwell plate, a plate, a tube, or a fluidic channel.

28. The method of any one of claims 1-27, wherein the one or more target polynucleotides are DNA.

29. The method of any one of claims 1-28, wherein the one or more target polynucleotides are a single-stranded polynucleotides.

30. The method of any one of claims 1-28, wherein the one or more target polynucleotides are double-strand polynucleotides.

31. The method of claim 30, wherein the method further comprises denaturing any polynucleotides in the sample prior to step (b) to generate single-strand polynucleotides.

32. The method of any one of claims 1-31, wherein a bisulfate conversion of the DNA sample occurred before step (a).

33. The method of any one of claims 1-27, wherein the one or more target polynucleotides are RNA.

34. The method of claim 33, wherein reverse transcriptase is contacted to the sample before step (b).

35. The method of any one of claims 1-34, wherein the target polynucleotides are linked to a barcode at their 5′ end, 3′ end, or at both ends.

36. The method of any one of claims 1-35, wherein a nucleic acid purification step is performed on the sample prior to step (a).

37. The method of any one of claims 1-35, wherein no nucleic acid purification step is performed on the sample prior to step (a).

38. The method of claim 37, wherein the step (a) comprises (i) annealing the targeting nucleic acid to the corresponding target polynucleotide in the sample, (ii) washing the solid surface to remove contaminates from the sample, (iii) contacting the solid surface with a reaction mix.

39. The method of any one of claims 1-38, wherein the sample comprises a reference sequence, and targeting nucleic acids for the reference sequence are immobilized to the solid surface.

40. The method of any one of claims 1-39, wherein the sample is diluted prior to the step (a).

41. The method of any one of claims 1-40, wherein a pool of samples is used in the step (a).

42. The method of any one of claim 1-41, wherein the amplification process is a semi-solid phase amplification with one end on the solid surface and the other end in suspension.

43. The method of any one of claim 1-41, wherein the amplification process is a solid phase amplification.

44. The method of any one of claims 1-41, wherein the amplification process occurs both on the solid phase and in suspension.

45. The method of claim 43 or 44, wherein the solid phase amplification process is a bridge amplification.

46. The method of any one of claims 1-45, wherein the amplification process is a stepwise thermal amplification.

47. The method of any one of claims 1-45, wherein the amplification process is a stepwise chemical amplification.

48. The method of claim 46 or 47, wherein the amplification process is PCR or RT-PCR.

49. The method of any one of claims 1-45, wherein the amplification process is an isothermal amplification.

50. The method of claim 49, wherein the isothermal amplification process is TMA, NASBA, LAMP, HIP, HDA, RPA, SDA, or rolling circle amplification.

51. The method of claim 49 or 50, wherein the step (b) occurs at an ambient temperature.

52. The method of claim 49 or 50, wherein the step (b) occurs at about 37° C. to about 42° C.

53. The method of any one of claim 1-50, wherein the step (b) occurs in the presence of heat generated by a living body.

54. The method of claim 53, wherein the living body is a human body.

55. The method of claim 49 or 50, wherein the step (b) occurs at human body temperature.

56. The method of claim 55, wherein the human body temperature is generated from the heat of a human body.

57. The method of any one of claims 49-56, wherein the step (b) occurs without a mechanical heating device.

58. The method of any one of claims 46-50 and 52, wherein heat induced by chemical exothermic reaction at the step (b).

59. The method of any one of claims 1-45, and 47, wherein the amplification is non-enzymatic amplification process.

60. The method of claim 59, wherein the non-enzymatic amplification process is TMSD-mediated HCR.

61. The method of any one of claims 1-60, wherein the clusters of amplicons are detected using naked eye, a phase microscope, IRIS, observable sediment formation, SPR, or electric conductivity.

62. The method of any one of claims 1-60, further comprising labeling the clusters of amplicons prior to the step (c).

63. The method of claim 62, wherein the clusters of amplicons are labeled using a DNA binding dye.

64. The method of claim 63, wherein the DNA binding dye is an intercalating dye or a groove binding dye.

65. The method of claim 63 or 64, wherein the DNA binding dye is SYTO-9, SYTO-13, SYTO-82, SYBR Green I, SYBR Gold, EvaGreen, PicoGreen, Ethidium Bromide, Acridine, Propidium Iodide, Crystal Violet, DAPI, 7-AAD, Hoechst, YOYO-1, DiYO-1, TOTO-1, DiTO-1, Hydroxystyryl-Quinolizinium Photoacid, or styryl dye.

66. The method of any one of claims 1-60, wherein the clusters of amplicons are labeled by incorporating a labeled nucleotide.

67. The method of claim 62, wherein the clusters of amplicons are labeled using a surface bound or suspended probe.

68. The method of claim 67, wherein the surface bound probe is in the same cluster as the targeting nucleic acid.

69. The method of claim 67 or 68, wherein the surface bound probe is bound by the 3′ end.

70. The method of any one of claims 67-69, wherein the probe is a taqman probe, molecular beacon, or scorpion.

71. The method of any one of claims 66-70, wherein the nucleotide or probe is labeled with biotin, DIG, dppz, Ruthenium(II) complex, gold, Crystal Violet, HRP+TMB, Alkaline phosphatase, palladium, platinum, magnetic nanoparticle, BSA-MnO2 NPs, graphene oxide, Carbon dots, or agents for electrochemical detection.

72. The method of any one of claims 62-71, wherein amplicons of adjacent clusters are labeled differently.

73. The method of any one of claims 62-71, wherein amplicons of different target polynucleotides are labeled the same but at pre-determined locations.

74. The method of any one of claims 62-73, wherein the labeled clusters of amplicons are detected by a scanner, a fluorescence microscope, or a camera, optionally wherein the camera is a cell phone camera.

75. The method of any one of claims 1-74, wherein the number of distinct clusters of amplicons for each target polynucleotide is counted at the step (c) to quantify the amount of each target polynucleotide in the sample.

76. A device comprising a first external surface, a second external surface that is different from the first external surface, an internal surface, a plurality of targeting nucleic acids immobilized to pre-determined locations on the internal surface, and a reaction chamber enclosed by the internal surface, wherein targeting nucleic acids specific for different target polynucleotides are immobilized at different predetermined locations on the internal surface.

77. The device of claim 76, wherein the device is substantially flat.

78. The device of claim 76 or 77, wherein the device has a surface/volume ratio larger than 1.3/mm.

79. The device of any one of claims 76 to 78, wherein the first external surface comprises a material that is able to conduct heat.

80. The device of any one of claims 76 to 79, wherein the second external surface is on the opposite side to the first external surface.

81. The device of any one of claims 76 to 80, wherein the second external surface comprise an insulating material.

82. The device of any one of claims 76 to 81, wherein at least one external surface is transparent.

83. The device of any one of claims 76 to 82, wherein the device further comprises a strap or a tape to fix the device to a human body.

84. The device of any one of claims 76 to 83, wherein the targeting nucleic acids comprise primers.

85. The device of claim 84, wherein only forward primers or reverse primers specific to the target polynucleotides are immobilized to the internal surface.

86. The device of claim 85, wherein the reaction chamber comprises the other primer that forms a primer pair with the immobilized forward primer or reverse primer.

87. The device of claim 84, wherein both forward and reverse primers specific to the target polynucleotides are immobilized to the internal surface.

88. The device of any one of claims 76 to 87, wherein the targeting nucleic acids specific for each target polynucleotide are uniformly immobilized to the predetermined location of the internal surface.

89. The device of any one of claims 76 to 87, wherein the plurality of targeting nucleic acids are immobilized as an array of targeting nucleic acid clusters, with each targeting nucleic acid cluster located at one of the predetermined locations on the internal surface.

90. The device of claim 89, wherein the targeting nucleic acid clusters are spatially separated from each other in space on the internal surface.

91. The device of claim 89 or 90, wherein multiple clusters of targeting nucleic acids for the same target polynucleotides are immobilized on the internal surface.

92. The device of claim 91, wherein the targeting nucleic acids comprise a set of nested primers for the same target polynucleotide.

93. The device of claim 92, wherein one cluster of primers spreads into another cluster of primers within the set of nested primers for the target polynucleotides.

94. The device of any one of claims 89-93, wherein each cluster of targeting nucleic acids comprises at least 2 copies of the same targeting nucleic acids.

95. The device of any one of claims 76-94, wherein the targeting nucleic acid is immobilized to the internal surface via 5′ end.

96. The device of any one of claims 76-95, wherein the targeting nucleic acid is covalently linked to the internal surface.

97. The device of any one of claims 76-95, wherein the targeting nucleic acid is non-covalently linked to the internal surface.

98. The device of claim 97, wherein the targeting nucleic acid is immobilized to the internal surface via passive absorption, streptavidin-biotin interaction, or hybridization.

99. The device of any one of claim 76-98, wherein the 3′end of the targeting nucleic acid is free to elongate.

100. The device of any one of claim 76-99, wherein each cluster comprise targeting nucleic acids at a density of at least 9/micron².

101. The device of any one of claims 76-100, wherein the targeting nucleic acid is an invading primer.

102. The device of claim 101, wherein the invading primer is LNA, PNA, PTO, ZNA, invader probe, or INA.

103. The device of any one of claims 76-102, wherein the device further comprises a reaction mix in the reaction chamber.

104. The device of claim 103, wherein the reaction mix comprises dNTPs, buffer, and at least one enzyme for isothermal amplification.

105. The device of claim 104, wherein the isothermal amplification is TMA, NASBA, LAMP, HIP, HDA, RPA, SDA, or rolling circle amplification.

106. The device of any one of claims 76-105, wherein the device is used for detecting presence of one or more target polynucleotides in a sample.

107. The device of any one of claims 76-106, wherein the device is used for quantifying the amount of one or more target polynucleotides in a sample.

108. The device of any one of claims 76-107, wherein the device is used at a body temperature.

109. The device of claim 106 or 107, wherein one or more target polynucleotides are detected using the naked eye or a cell phone camera.

110. The device of any one of claims 76-109, wherein the sample comprises a reference sequence and targeting nucleic acids for the reference sequence are immobilized to the inner surface.

111. The device of any one of claims 76-110, wherein the targeting nucleic acids are positioned in a specific pattern according to a test serial number.

112. The device of any one of claims 76-111, wherein the internal surface is a 3D polymer.
